(19) United States
(12) Patent Application Publication
(10) Pub. No.: US 2022/0230708 A1
(43) Pub. Date: Jul. 21, 2022

(54) METHOD FOR DETECTING OUTLIER OF THEORETICAL MASSES

(71) Applicant: SHIMADZU CORPORATION, Kyoto-shi, Kyoto (JP)

(72) Inventor: Tatsuki OKUBO, Kyoto-shi, Kyoto (JP)

(73) Assignee: SHIMADZU CORPORATION, Kyoto-shi, Kyoto (JP)

(21) Appl. No.: 17/607,080

(22) PCT Filed: Feb. 20, 2020
(86) PCT No.: PCT/JP2020/006834

§ 371 Date: Oct. 28, 2021

(30) Foreign Application Priority Data
May 10, 2019 (JP) 2019-089764

(51) Int. Cl. G16B 40/00

(52) U.S. Cl. CPC G16B 40/00

(57) ABSTRACT

A representative value is decided from a theoretical mass group which is a set of theoretical masses regarding the same type of protein of a plurality of microorganisms (step S1), a reference sequence which is an amino acid sequence or a base sequence corresponding to the representative value is specified (step S2), an editing distance between an amino acid sequence or a base sequence corresponding to each of the theoretical masses included in the theoretical mass group and the reference sequence is calculated (step S3), and a theoretical mass corresponding to an amino acid sequence or a base sequence of which the editing distance is equal to or greater than a predetermined threshold value is decided, as an outlier, among the theoretical masses included in the theoretical mass group (step S4).

### TECHNICAL FIELD

The present invention relates to a method for detecting an outlier of theoretical masses.

### BACKGROUND ART

In recent years, a microorganism identification method using mass spectrometry has been developed (see, for example, Patent Literature 1). In this method, first, a solution containing proteins extracted from a test microorganism, a suspension of the test microorganism, or the like is analyzed by a mass spectrometer using a soft ionization method such as matrix-assisted laser desorption ionization mass spectrometry (MALDI-MS). The “soft” ionization method refers to an ionization method in which a high-molecular-weight compound is hardly decomposed. A microorganism species or a microorganism strain of the test microorganism is specified by collating an obtained mass spectrum with amass spectrum of the known microorganism.

In the microorganism identification method using mass spectrometry as described above, microorganisms are identified by focusing on mass spectrum peaks having different masses between species or strains of microorganisms. Such a mass spectrum peak is called a marker peak, and for example, a peak or peaks derived from a protein having relatively high preservability such as a ribosomal protein is used as a marker peak.

In order to identify unknown microorganisms based on a mass of the marker peak, it is necessary to specify the mass of the marker peak for each species or each strain of the microorganism in advance, and store these pieces of information in a database. However, it is not realistic to obtain a large number of microorganisms of different species or strains, and to actually perform mass spectrometry for each microorganism to measure the mass of the marker peak. Thus, it is considered that a theoretical mass (calculated mass) of the marker peak is calculated based on amino acid sequence data or base sequence data (hereinafter, referred to as “amino acid sequence data or the like”) of various microorganisms recorded in a public database (for example, GenBank, EMBL, DDBJ, or the like) and the calculated mass is used for the identification of the unknown microorganism by the mass spectrometry as described above.

### CITATION LIST

### Patent Literature

Patent Literature 1: WO 2017/168742 A

### SUMMARY OF INVENTION

### Technical Problem

Value of theoretical mass calculated from the amino acid sequence data or the like recorded in the public database may have a large variation between microbial strains even though the theoretical mass is derived from the same type of protein. When a calculated value of the theoretical mass is greatly different from another value, there is a high possibility that an error is included in the amino acid sequence data or the like (which is caused by a sequencing error or the like) on which the calculation of the theoretical mass is based. Thus, when such a theoretical mass is adopted as the mass of the marker peak, there is a concern that accuracy of the microorganism identification is inadequate. Accordingly, it is necessary to remove an outlier (that is, data having an abnormal value which harms the accuracy of the identification) by using some criterion, but there is a problem that an appropriate criterion for removing the outlier is not determined.

The present invention has been made in view of the above points, and an object is to provide a method for appropriately detecting an outlier from a data set including theoretical mass data related to the same type of protein of a plurality of microorganisms.

### Solution to Problem

A method for detecting an outlier of theoretical masses according to the present invention is achieved to solve the problem, the method including: deciding a representative value from a theoretical mass group which is a set of theoretical masses regarding the same type of protein of a plurality of microorganisms, specifying a reference sequence which is an amino acid sequence or a base sequence corresponding to the representative value; calculating an editing distance between an amino acid sequence or a base sequence corresponding to each of the theoretical masses included in the theoretical mass group and the reference sequence; and deciding, as an outlier, a theoretical mass corresponding to an amino acid sequence or a base sequence of which the editing distance is equal to or greater than a predetermined threshold value among the theoretical masses included in the theoretical mass group.

### Advantageous Effects of Invention

According to the method for detecting an outlier of theoretical masses according to the present invention, it is possible to appropriately detect an outlier from a data set including theoretical mass data regarding the same type of protein of a plurality of microorganisms.

### BRIEF DESCRIPTION OF DRAWINGS

FIG. 1 is a block diagram illustrating a configuration of main parts of a system including a theoretical mass outlier detection device according to an embodiment of the present invention.

FIG. 2 is a flowchart illustrating a flow of processing in the theoretical mass outlier detection device.

FIG. 3 is a diagram illustrating an outlier detection result in an example.

FIG. 4 is a diagram illustrating amino acid sequences corresponding to sequence patterns A to F in FIG. 3.

### DESCRIPTION OF EMBODIMENTS

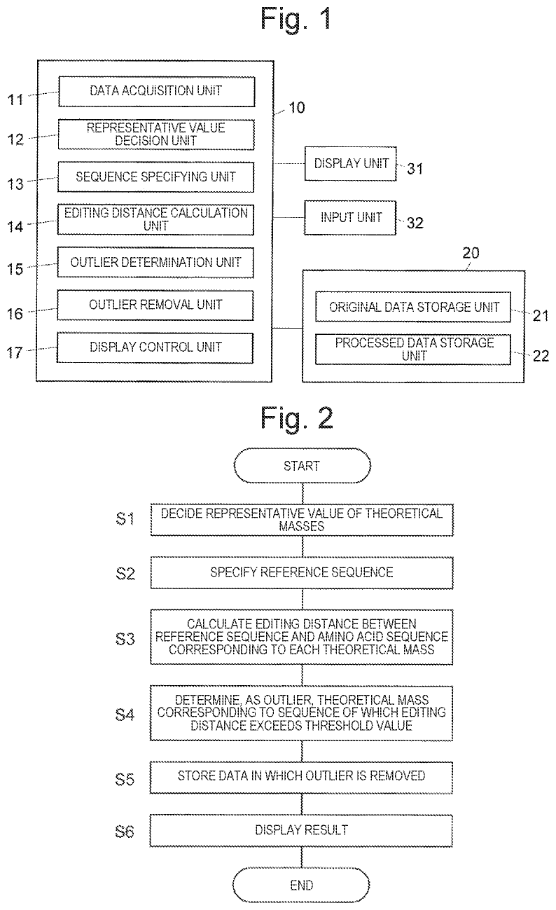

Hereinafter, an embodiment of the present invention will be described with reference to the drawings. FIG. 1 is a block diagram illustrating a configuration of main parts of a system including a theoretical mass outlier detection device (hereinafter, referred to as an “outlier detection device 10”) according to the present embodiment. The system includes an outlier detection device 10, a storage unit 20, a display unit 31, and an input unit 32.

The outlier detection device 10 includes, as functional blocks, a data acquisition unit 11, a representative value decision unit 12, a sequence specifying unit 13, an editing distance calculation unit 14, an outlier determination unit 15, an outlier removal unit 16, and a display control unit 17. The outlier detection device 10 is embodied by using a personal computer including a CPU, a memory, and the like as hardware resources and executing dedicated software installed in the personal computer by the CPU.

The storage unit 20 includes an original data storage unit 21 that stores theoretical mass data (original data) as a target of outlier detection, and a processed data storage unit 22 that stores data (processed data) obtained by removing an outlier from the original data. The storage unit 20 can be realized by a mass storage device such as a hard disk drive (HDD) or a solid state drive (SSD) built in or externally attached to the personal computer constituting the outlier detection device 10.

The display unit 31 includes a liquid crystal display device or the like, and the input unit 32 includes a keyboard and a pointing device such as a mouse, and both the units are connected to the personal computer constituting the outlier detection device 10.

FIG. 2 is a flowchart illustrating an execution procedure of the outlier detection by the outlier detection device 10 according to the present embodiment. When the outlier is detected, a plurality of theoretical masses (regarding the same type of protein of a plurality of microorganisms, and corresponds to a “theoretical mass group” in the present invention) as the target of the outlier detection, an amino acid sequence that is the basis of each theoretical mass, and information regarding the origin (which protein of which microorganism strain the theoretical mass relates to) are stored in association with each other in the original data storage unit 21 in advance. The plurality of theoretical masses can be obtained by acquiring the amino acid sequence of the same type of protein (for example, any of ribosomal proteins) in a plurality of microbial strains from an existing database (for example, public databases such as GenBank, EMBL, or DDBJ), obtaining a calculated molecular weight of each protein by calculation from the amino acid sequence, and converting the calculated molecular weight into an ion mass of each protein. It is known that when a biological sample is analyzed by MALDI-MS, molecular weight-related ions such as [M+H]⁺ (M is a molecule and H is a hydrogen atom), [M−H]⁻, or [M+Na]⁺ (Na is a sodium atom) are mainly detected. Accordingly, when mass spectrometry conditions are determined, the conversion from the calculated molecular weight to the ion mass can be easily performed. When calculated molecular weights of proteins contained in various microbial strains are recorded in the existing database, the theoretical mass may be calculated by using the calculated molecular weights.

In the outlier detection by the outlier detection device 10 according to the present embodiment, first, the representative value decision unit 12 reads out the plurality of theoretical masses M1, M2, . . . , and Mn (n is a natural number) stored in the original data storage unit 21 by accessing the storage unit 20 via the data acquisition unit 11, specifies a mode value Mf thereof, and decides the mode value Mf as the representative value (step S1). Subsequently, the sequence specifying unit 13 specifies an amino acid sequence (hereinafter, referred to as “reference sequence Ar”) corresponding to the mode value Mf while referring to the original data storage unit 21 by accessing the storage unit 20 via the data acquisition unit 1 (step S2). Subsequently, the editing distance calculation unit 14 reads out amino acid sequences A1, A2, . . . and An corresponding to the plurality of theoretical masses M1, M2, . . . and Mn from the original data storage unit 21 by accessing the storage unit 20 via the data acquisition unit 11, and calculates editing distances d1, d2, . . . , and dn between the amino acid sequences A1, A2, . . . , and An and the reference sequence Ar (step S3). Here, the editing distance (Levenshtein distance) is a value indicating how much two character strings are different from each other, and specifically, is defined as the minimum number of procedures required to transform one character string into the other character string by insertion, deletion, or substitution of one character.

Subsequently, the outlier determination unit 15 determines, for each of the editing distances d1, d2, . . . , and dn obtained in step S3 for each of the amino acid sequences A1, A2, . . . and An, whether the value exceeds a predetermined threshold value dt, and determines that the theoretical mass corresponding to the amino acid sequence is the outlier when the value exceeds the threshold value dt (step S4). The threshold value dt is set in advance by a user via the input unit 32 and is stored in the storage unit 20, for example. Thereafter, the outlier removal unit 16 acquires a data set (that is, a plurality of theoretical masses as targets of the outlier detection, an amino acid sequence on which each theoretical mass is based, and information regarding the origin thereof) stored in the original data storage unit 21 by accessing the storage unit 20 via the data acquisition unit 11, removes data regarding the theoretical mass determined to be the outlier in step S4 from the data set, and stores the data set after removal in the processed data storage unit 22 (step S5). When the series of processing are completed, the data regarding the theoretical mass determined to be the outlier is displayed on the display unit 31 under the control of the display control unit 17 and is presented to the user (step S6).

As described above, in the outlier detection device according to the present embodiment, the outlier of the theoretical mass is detected based on a difference between the reference sequence and each amino acid sequence. Thus, it is possible to perform appropriate outlier detection in consideration of amino acid sequence data. Accordingly, the remaining theoretical mass (that is, the data set stored in the processed data storage unit 22) is derived from amino acid sequences similar to each other (that is, highly reliable amino acid sequences). Thus, it is possible to perform highly accurate microbial strain identification by adopting these theoretical masses as a mass of a marker peak of each of the microbial strains and collating a mass spectrometry result of a test microorganism with the mass of the marker peak of each of the microbial strains. As described above, the outlier detection device according to the present embodiment decides the representative value based on the theoretical mass that is numerical data and uses the amino acid sequence corresponding to the representative value as the reference sequence. Thus, for example, it is possible to suppress a calculation amount and improve a processing speed as compared with a case where the amino acid sequences that are character string data are compared with each other and the sequence having a highest appearance frequency is used as the reference sequence.

The embodiment for carrying out the present invention has been described above with reference to specific examples. The present invention is not limited to the above-described embodiment, and modifications can be appropriately made within the scope of the gist of the present invention. For example, in the above embodiment, the representative value decision unit 12 decides the mode value among the plurality of theoretical masses as the representative value. A median value may be used as the representative value instead of the mode value.

In the above embodiment, the sequence specifying unit 13 decides the amino acid sequence corresponding to the representative value as the reference sequence and the editing distance calculation unit 14 obtains the editing distances between the reference sequence and the amino acid sequences corresponding to the plurality of theoretical masses. Alternatively, the sequence specifying unit 13 may decide a base sequence corresponding to the representative value as the reference sequence, and the editing distance calculation unit 14 may obtain editing distances between the reference sequence and the base sequences corresponding to the plurality of theoretical masses.

In the above embodiment, the storage unit 20 is built in or externally attached to the personal computer constituting the outlier detection device 10. The storage unit 20 may be provided in another computer connected to the personal computer constituting the outlier detection device 10 directly or via the Internet, a local area network (LAN), or the like. In this case, the data acquisition unit 11 can access the storage unit 20 via the Internet or a LAN.

In the above embodiment, a program for the outlier detection is installed in advance in the computer. The program may be stored in a computer-readable recording medium and may be provided.

### Example

Amino acid sequences of a ribosomal protein L15 of 89 strains of Cutibacterium acnes were obtained from a public database, theoretical masses were calculated, and an outlier was detected from the theoretical masses.

The theoretical masses were distributed in a range of 15347.58 to 20635.62 with a mode value of 15384.69. Among the amino acid sequences of the 89 strains, the amino acid sequence corresponding to the mode value was used as the reference sequence, and editing distances between the reference sequence and the amino acid sequences of the 89 strains were calculated. A threshold value for the outlier determination was set to 2, and the theoretical mass of the strain having the editing distance exceeding the threshold value was determined as the outlier.

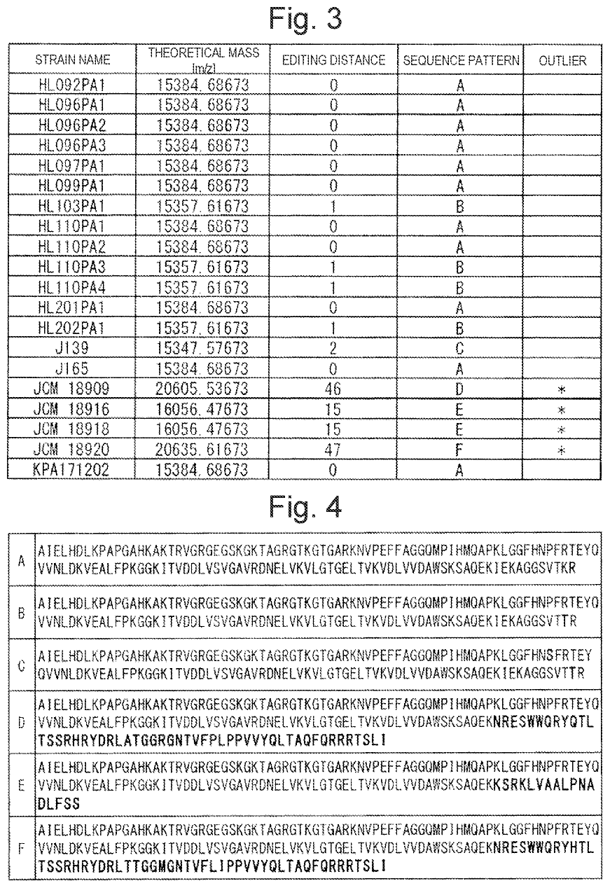

Detection results of the outlier are represented in FIG. 3. For the sake of simplicity, only results for 20 strains among the 89 strains are represented here. In the figure, a fourth row from the left represents an amino acid sequence pattern of the ribosomal protein L15 of each strain. Amino acid sequences corresponding to amino acid sequence patterns A to F are represented in FIG. 4. In the amino acid sequence patterns represented in FIG. 4, a sequence of pattern A is an amino acid sequence corresponding to the mode value (that is, a reference sequence). Editing distances between the amino acid sequence of the reference sequence and the amino acid sequences of the ribosomal protein L15 of the strains are as represented in a third row from the left in FIG. 3, and the strains having the editing distance exceeding 2 (that is, the strains of which the theoretical masses are determined to be the outlier) were 4 strains denoted by * in the same figure.

### [Aspects]

It is understood by those skilled in the art that the exemplary embodiments described above are specific examples of the following aspects.

(First aspect) A method for detecting an outlier of theoretical masses according to an aspect includes: deciding a representative value from a theoretical mass group which is a set of theoretical masses regarding the same type of protein of a plurality of microorganisms; specifying a reference sequence which is an amino acid sequence or a base sequence corresponding to the representative value; calculating an editing distance between an amino acid sequence or a base sequence corresponding to each of the theoretical masses included in the theoretical mass group and the reference sequence; and deciding, as an outlier, a theoretical mass corresponding to an amino acid sequence or a base sequence of which the editing distance is equal to or greater than a predetermined threshold value among the theoretical masses included in the theoretical mass group.

According to the method for detecting an outlier of theoretical masses described in the first aspect, it is possible to detect the outlier of the theoretical mass in consideration of the amino acid sequence or the base sequence. Thus, highly reliable outlier detection can be realized.

(Second aspect) In the method for detecting an outlier of theoretical masses according to the first aspect, the representative value may be a mode value.

The amino acid sequence or the base sequence corresponding to the mode value of the theoretical mass can be said to be a sequence having a highest appearance frequency among the amino acid sequences or the base sequences corresponding to the theoretical masses included in the theoretical mass group. Thus, the sequence having the highest appearance frequency can be set as the reference sequence by setting the mode value as the representative value of the theoretical masses, and more appropriate outlier determination can be realized by performing the outlier determination based on the distance (editing distance) from the reference sequence.

(Third aspect) In the method for detecting an outlier of theoretical masses according to the first or second aspect, the same type of protein may be a ribosomal protein.

(Fourth aspect) A program according to an aspect causes a computer to execute the method for detecting an outlier of theoretical masses according to any one of the first to third aspects.

(Fifth aspect) A non-transitory computer readable medium according to an aspect has the program according to the fourth aspect stored thereon.

### REFERENCE SIGNS LIST

10 . . . Outlier Detection Device
11 . . . Data Acquisition Unit
12 . . . Representative Value Decision Unit
13 . . . Sequence Specifying Unit
14 . . . Editing Distance Calculation Unit
15 . . . Outlier Determination Unit
16 . . . Outlier Removal Unit
17 . . . Display Control Unit
20 . . . Storage Unit
21 . . . Original Data Storage Unit
22 . . . Processed Data Storage Unit
31 . . . Display Unit
32 . . . Input Unit

1. A method for detecting an outlier of theoretical masses, the method comprising:
deciding a representative value from a theoretical mass group which is a set of theoretical masses regarding the same type of protein of a plurality of microorganisms;
specifying a reference sequence which is an amino acid sequence or a base sequence corresponding to the representative value;
calculating an editing distance between an amino acid sequence or a base sequence corresponding to each of the theoretical masses included in the theoretical mass group and the reference sequence; and
deciding, as an outlier, a theoretical mass corresponding to an amino acid sequence or a base sequence of which the editing distance is equal to or greater than a predetermined threshold value among the theoretical masses included in the theoretical mass group.

2. The method for detecting an outlier of theoretical masses according to claim 1, wherein the representative value is a mode value.

3. The method for detecting an outlier of theoretical masses according to claim 1, wherein the same type of protein is a ribosomal protein.

4. A non-transitory computer-readable medium recording a program causing a computer to execute the method for detecting an outlier of theoretical masses according to claim 1.
